(19) United States
(12) Patent Application Publication
(10) Pub. No.: US 2025/0057436 A1
(43) Pub. Date: Feb. 20, 2025

(54) DATA PROCESSING DEVICE, DATA PROCESSING METHOD, AND MAGNETIC RESONANCE IMAGING APPARATUS

(71) Applicant: FUJIFILM Corporation, Tokyo (JP)

(72) Inventor: Shohei HATORI, Chiba (JP)

(73) Assignee: FUJIFILM Corporation, Tokyo (JP)

(21) Appl. No.: 18/793,924

(22) Filed: Aug. 4, 2024

(30) Foreign Application Priority Data
Aug. 15, 2023 (JP) 2023-132294

(51) Int. Cl. A61B 5/055; A61B 5/00; A61B 5/318

(52) U.S. Cl. CPC A61B 5/055; A61B 5/318; A61B 5/7285; A61B 5/742

(57) ABSTRACT

A data processing device includes: a reconstruction unit that reconstructs a plurality of images of a heart along a time series based on nuclear magnetic resonance (NMR) signals acquired by a magnetic resonance imaging apparatus (MRI apparatus); a time-phase detection unit that detects one or more cardiac rest phases based on the plurality of images reconstructed by the reconstruction unit; a display control unit that displays the cardiac rest phase on a display unit; an operation unit that accepts an input of a correction operation of the cardiac rest phase; and a time-phase correction unit that corrects the cardiac rest phase based on the correction operation input to the operation unit.

### CROSS-REFERENCE TO RELATED APPLICATION

The present application claims priority under 35 U.S.C § 119 (a) to Japanese Patent Application No. 2023-132294 filed on Aug. 15, 2023, which is hereby expressly incorporated by reference, in its entirety, into the present application.

### BACKGROUND OF THE INVENTION

### 1. Field of the Invention

The present invention relates to a data processing device and a data processing method of a magnetic resonance imaging apparatus, and a magnetic resonance imaging apparatus comprising the data processing device.

### 2. Description of the Related Art

A magnetic resonance imaging apparatus [also referred to as an MRI apparatus] magnetically excites nuclear spins of a subject placed in a static magnetic field with a radio frequency (RF) pulse at a Larmor frequency and reconstructs an image from a nuclear magnetic resonance (NMR) signal generated by the excitation. In a case where the magnetic resonance imaging apparatus is used for a cardiac examination, the magnetic resonance imaging apparatus performs electrocardiogram gated imaging of acquiring the NMR signal of a heart in synchronization with an electrocardiogram waveform of the heart, thereby obtaining an image of the heart with less artifacts caused by pulsation.

A magnetic resonance imaging apparatus described in U.S. Pat. No. 11,119,176B executes a pre-scan (acquisition of MR signals) for two heartbeats in synchronization with an electrocardiogram waveform and detects end-diastolic and end-systolic cardiac rest phases (a start phase and an end phase of a cardiac rest period) for one heartbeat of a heart based on NMR signals obtained in the pre-scan. The magnetic resonance imaging apparatus described in U.S. Pat. No. 11,119,176B sets an imaging time phase (such as a delay time from an R-wave of the electrocardiogram waveform) during the main imaging of cardiac MRI based on detection results of the cardiac rest phase in the pre-scan and pre-set user settings.

### SUMMARY OF THE INVENTION

Meanwhile, the cardiac rest phase detected in the pre-scan of the magnetic resonance imaging apparatus described in U.S. Pat. No. 11,119,176B may be different from an actual cardiac rest phase. In this case, in the magnetic resonance imaging apparatus described in U.S. Pat. No. 11,119,176B, it is not possible to easily execute correction (adjustment) of the cardiac rest phase detected in the pre-scan.

The present invention has been made in view of such circumstances, and an object of the present invention is to provide a data processing device and a data processing method capable of easily executing correction of a detection result of a cardiac rest phase in a case where detection of the cardiac rest phase is executed, a magnetic resonance imaging apparatus comprising the data processing device.

According to a first aspect of the present invention, there is provided a data processing device of a magnetic resonance imaging apparatus that acquires nuclear magnetic resonance signals of a heart in synchronization with an electrocardiogram waveform for a period of one or more heartbeats, the data processing device comprising: a reconstruction unit that reconstructs a plurality of images of the heart along a time series based on the nuclear magnetic resonance signals acquired by the magnetic resonance imaging apparatus; a time-phase detection unit that detects one or more cardiac rest phases based on the plurality of images reconstructed by the reconstruction unit; a display control unit that displays the cardiac rest phase on a display unit; an operation unit that accepts an input of a correction operation of the cardiac rest phase; and a time-phase correction unit that corrects the cardiac rest phase based on the correction operation input to the operation unit.

With this data processing device, a user can easily execute correction (adjustment) of the cardiac rest phase based on the cardiac rest phase displayed on the display unit.

According to a second aspect of the present invention, in the data processing device described in the first aspect, a parameter setting unit that sets a scan parameter of the magnetic resonance imaging apparatus based on the cardiac rest phase is further provided, and the parameter setting unit sets, in a case where the correction operation is input to the operation unit, the scan parameter based on the cardiac rest phase corrected by the time-phase correction unit. As a result, the scan parameter can be set based on the cardiac rest phase corrected in the correction operation.

According to a third aspect of the present invention, in the data processing device described in the second aspect, the operation unit is configured to accept an input of a re-correction operation of the cardiac rest phase that has been corrected in the correction operation, the time-phase correction unit re-corrects, in a case where the re-correction operation is input to the operation unit, the cardiac rest phase based on the re-correction operation, and the parameter setting unit sets, in a case where the re-correction operation is input to the operation unit, the scan parameter based on the cardiac rest phase re-corrected by the time-phase correction unit. As a result, the cardiac rest phase can be re-corrected and reflected in the scan parameter even after scan parameter settings.

According to a fourth aspect of the present invention, in the data processing device described in the first to third aspects, the time-phase detection unit detects a first cardiac rest phase indicating an end-systolic cardiac rest phase of the heart and a second cardiac rest phase indicating an end-diastolic cardiac rest phase of the heart, the display control unit displays the first cardiac rest phase and the second cardiac rest phase on the display unit in an identifiable manner, the operation unit accepts inputs of the correction operation of the first cardiac rest phase and the correction operation of the second cardiac rest phase, and the time-phase correction unit corrects the first cardiac rest phase in a case where the correction operation of the first cardiac rest phase is input to the operation unit, and corrects the second cardiac rest phase in a case where the correction operation of the second cardiac rest phase is input to the operation unit. As a result, the user can easily correct the first cardiac rest phase of the end-systole of the heart and the second cardiac rest phase of the end-diastole of the heart.

According to a fifth aspect of the present invention, in the data processing device described in the fourth aspect, a parameter setting unit that sets a scan parameter of the magnetic resonance imaging apparatus based on the cardiac rest phase is further provided, the operation unit is configured to accept an input of a time-phase selection operation of selecting any one of the first cardiac rest phase or the second cardiac rest phase, and the parameter setting unit sets, in a case where the time-phase selection operation is input to the operation unit, the scan parameter based on the one selected in the time-phase selection operation. As a result, the scan parameter can be set based on any one of the first cardiac rest phase or the second cardiac rest phase.

According to a sixth aspect of the present invention, in the data processing device described in the fourth aspect, a parameter setting unit that sets a scan parameter of the magnetic resonance imaging apparatus based on the cardiac rest phase is further provided, and the parameter setting unit selects any one of the first cardiac rest phase or the second cardiac rest phase in accordance with a predetermined recommendation condition and sets the scan parameter based on the selected one. As a result, the scan parameter can be set based on the one recommended in the recommendation condition among the first cardiac rest phase and the second cardiac rest phase.

According to a seventh aspect of the present invention, in the data processing device described in any one of the first to sixth aspects, the display control unit displays the cardiac rest phase on the display unit in accordance with a time axis.

According to an eighth aspect of the present invention, in the data processing device described in the seventh aspect, the display control unit displays, on the display unit, a time-phase display region including the cardiac rest phase and the time axis, and an image display region for selectively displaying any of the plurality of images reconstructed by the reconstruction unit, the operation unit is configured to accept an input of a time designation operation of designating a certain time of the time axis, and the display control unit displays, in a case where the time designation operation is input to the operation unit, an image corresponding to the time designated in the time designation operation among the plurality of images reconstructed by the reconstruction unit, in the image display region. As a result, the image corresponding to the cardiac rest phase detected by the time-phase detection unit can be displayed on the display unit, so that the user can determine whether or not the cardiac rest phase detected by the time-phase detection unit is different from the actual cardiac rest phase.

According to a ninth aspect of the present invention, in the data processing device described in the seventh or eighth aspect, the display control unit displays, on the display unit, a time-phase display region including the cardiac rest phase and the time axis, and an image display region for selectively displaying any of the plurality of images reconstructed by the reconstruction unit, the operation unit is configured to accept an input of an image selection operation of selecting the image to be displayed in the image display region, and the display control unit displays, in a case where the image selection operation is input to the operation unit, the image selected in the image selection operation in the image display region and displays a time phase corresponding to the image selected in the image selection operation on the time axis in the time-phase display region. As a result, the time phase corresponding to the image of the cardiac rest period selected in the image selection operation can be displayed on the time axis, so that the user can understand the actual cardiac rest phase.

According to a tenth aspect of the present invention, in the data processing device described in any one of the first to ninth aspects, a standard value decision unit that decides on, based on a heart rate obtained from the electrocardiogram waveform, a standard value of the cardiac rest phase corresponding to the heart rate; and an accuracy calculation unit that calculates accuracy of the cardiac rest phase detected by the time-phase detection unit based on the standard value decided on by the standard value decision unit are further provided, and the display control unit displays a calculation result of the accuracy calculation unit on the display unit. As a result, even in a case where the user has less experience, the user can easily understand the accuracy of the cardiac rest phase.

According to an eleventh aspect of the present invention, in the data processing device described in any one of the first to tenth aspects, the reconstruction unit reconstructs the plurality of images at regular time intervals.

According to a twelfth aspect of the present invention, in the data processing device described in any one of the first to eleventh aspects, the time-phase detection unit calculates a representative value of signal values of the image for each of the plurality of images reconstructed by the reconstruction unit to detect the cardiac rest phase based on the representative value for each image along the time series.

According to a thirteenth aspect of the present invention, there is provided a magnetic resonance imaging apparatus comprising: a measurement control unit that acquires nuclear magnetic resonance signals of a heart in synchronization with an electrocardiogram waveform for a period of one or more heartbeats; and the data processing device according to any one of the first to twelfth aspects.

According to a fourteenth aspect of the present invention, there is provided a data processing method of a magnetic resonance imaging apparatus that acquires nuclear magnetic resonance signals of a heart in synchronization with an electrocardiogram waveform for a period of one or more heartbeats, the data processing method comprising: a reconstruction step of reconstructing a plurality of images of the heart along a time series based on the nuclear magnetic resonance signals acquired by the magnetic resonance imaging apparatus; a time-phase detection step of detecting one or more cardiac rest phases based on the plurality of images reconstructed in the reconstruction step; a display control step of displaying the cardiac rest phase on a display unit; an input acceptance step of accepting an input of a correction operation of the cardiac rest phase; and a time-phase correction step of correcting the cardiac rest phase based on the correction operation accepted in the input acceptance step.

According to the present invention, it is possible to easily execute the correction of the detection result of the cardiac rest phase in a case where the detection of the cardiac rest phase is executed.

### BRIEF DESCRIPTION OF THE DRAWINGS

FIG. 1 is a perspective view of a magnetic resonance imaging apparatus (MRI apparatus).

FIG. 2 is a functional block diagram of a control device of a first embodiment.

FIG. 3 is an explanatory diagram showing an example of signal value detection processing by a time-phase detection unit.

FIG. 4 is a graph showing a relationship between a time phase (horizontal axis) of each image of a time series image group spanning one heartbeat of a heart and a signal value (vertical axis) of each image.

FIG. 5 is an explanatory diagram illustrating an example of rest phase detection processing by the time-phase detection unit.

FIG. 6 is an explanatory diagram showing an example of a time-phase setting UI screen of the first embodiment.

FIG. 7 is an explanatory diagram showing an example of the time-phase setting UI screen after a correction operation.

FIGS. 8A to 8C are explanatory diagrams showing examples of a scan parameter.

FIG. 9 is a flowchart showing a data processing method of the MRI apparatus of the first embodiment, particularly a flow of scan parameter settings.

FIG. 10 is a flowchart showing a flow of re-correction operations of the first cardiac rest phase and the second cardiac rest phase after the scan parameter settings.

FIG. 11 is a functional block diagram of a control device of an MRI apparatus of a second embodiment.

FIG. 12 is an explanatory diagram showing an example of a time-phase setting UI screen of the second embodiment.

FIG. 13 is a flowchart showing a data processing method of the MRI apparatus of the second embodiment, particularly a flow of scan parameter settings.

### DESCRIPTION OF THE PREFERRED EMBODIMENTS

### First Embodiment

### Overall Configuration of MRI Apparatus

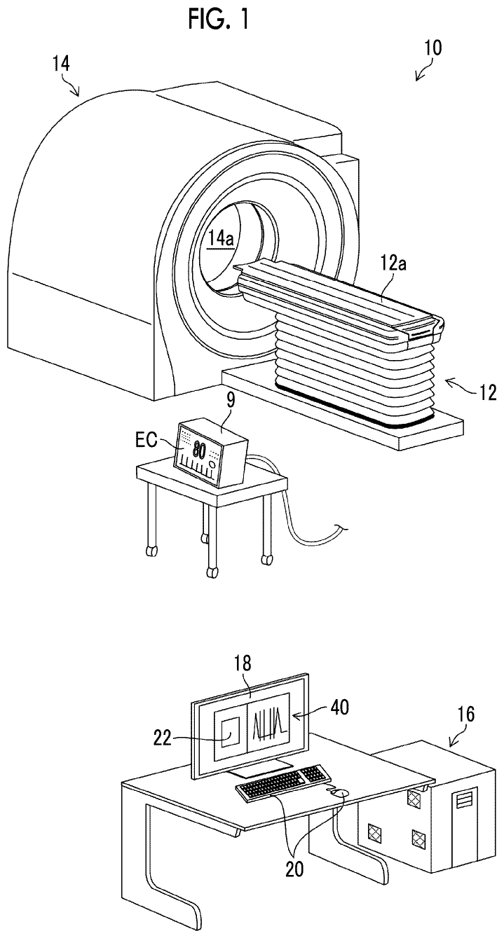

FIG. 1 is a perspective view of a magnetic resonance imaging apparatus 10 (MRI apparatus). As shown in FIG. 1, the MRI apparatus 10 is combined with an electrocardiograph 9 to execute a cardiac MRI examination, that is, to generate an image 22 of a heart. In the present embodiment, as the image 22 of the heart, a short-axis sectional image that allows observation of the left ventricle of the heart in a sliced manner is generated, but a long-axis sectional image that allows observation of the entire left ventricle of the heart or both the atria and both the ventricles of the heart may be generated.

The electrocardiograph 9 is connected to electrodes (not shown) attached to a subject, and continuously measures an electrocardiogram waveform EC of the heart of the subject and continuously outputs the electrocardiogram waveform EC to a control device 16 of the MRI apparatus 10.

The MRI apparatus 10 comprises a patient table 12, a measurement unit 14, the control device 16, a display unit 18, and an operation unit 20.

The patient table 12 comprises a tabletop 12a on which the subject (patient) who undergoes the cardiac MRI examination is placed. The tabletop 12a is movable in an up-down direction, in a direction of entry (forward direction) into an imaging space 14a of the measurement unit 14, and in a direction of exit (backward direction) from the imaging space 14a by a drive device (not shown).

Although not shown, the measurement unit 14 comprises a static magnetic field generation magnet, a gradient magnetic field coil, a transmission coil, a reception coil, and the like. The static magnetic field generation magnet generates a static magnetic field in the imaging space 14a. The gradient magnetic field coil provides a magnetic field gradient with respect to the static magnetic field generated by the static magnetic field generation magnet. The transmission coil generates a high-frequency magnetic field in a measurement region (heart) of the subject. The reception coil receives an NMR signal generated from the measurement region of the subject. Since the detailed configuration of each unit of the measurement unit 14 is a known technology, detailed descriptions will be omitted here. In the cardiac MRI examination, the measurement unit 14 applies a high-frequency magnetic field to the heart of the subject under the control of the control device 16 to be described below, acquires the NMR signal generated from the heart by the application of the high-frequency magnetic field, and outputs the NMR signal to the control device 16.

The electrocardiograph 9, the measurement unit 14, the display unit 18, and the operation unit 20 are connected to the control device 16. The control device 16 controls the measurement unit 14 based on the electrocardiogram waveform EC input from the electrocardiograph execute MRI imaging (electrocardiogram gated imaging) of acquiring the NMR signal of the heart in a state of being synchronized with the electrocardiogram waveform EC. In addition, the control device 16 functions as the data processing device of the embodiment of the present invention to execute the reconstruction of the image 22 of the heart based on the NMR signal acquired from the measurement unit 14, the detection of a cardiac rest phase, the display and the acceptance of a correction operation of the cardiac rest phase, and scan parameter settings of the MRI apparatus 10.

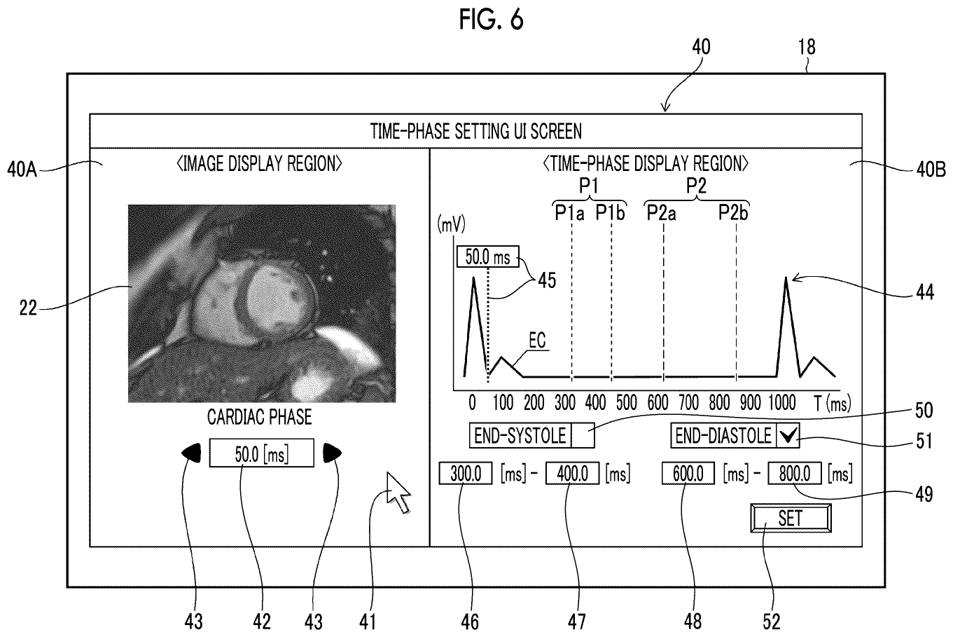

On the display unit 18, under the control of the control device 16, an operation setting screen for MRI imaging, the image 22 (diagnostic image) reconstructed by the control device 16, a time-phase setting UI screen 40, which is a user interface (UI) screen, and the like are displayed. The time-phase setting UI screen 40, which will be described in detail below, is a screen used for the correction operation of the cardiac rest phase and the scan parameter settings of the MRI apparatus 10 and includes the image 22 and a detection result of the cardiac rest phase (refer to FIG. 6).

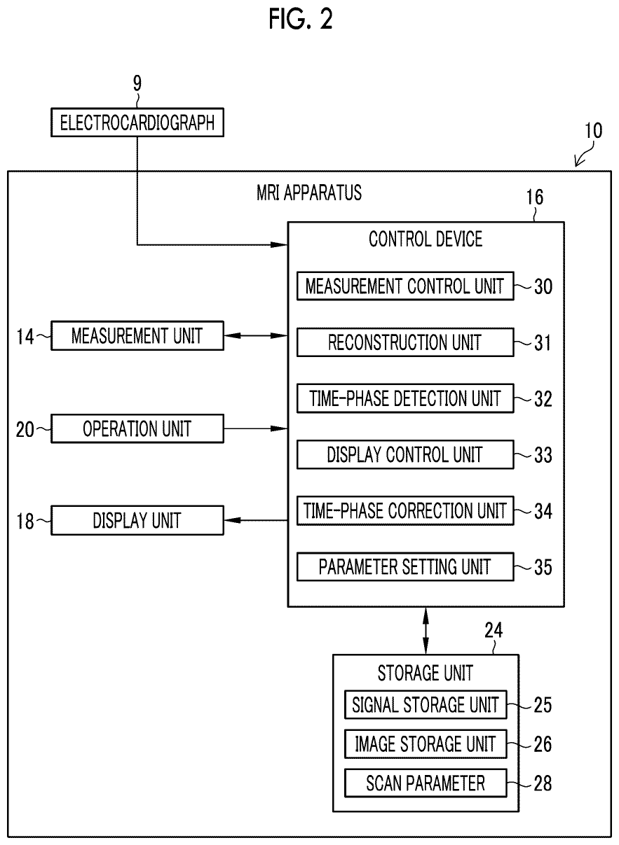

The operation unit 20 is composed of, for example, a keyboard and a mouse. The operation unit 20 accepts inputs of various operations by a user (examiner) of the MRI apparatus 10. The various operations include not only a start operation of the MRI imaging but also, as will be described in detail, various operations (a correction operation, a re-correction operation, a time-phase selection operation, a time designation operation, and an image selection operation) with respect to the time-phase setting UI screen 40. In addition, the start operation of the MRI imaging includes a start operation of cine imaging of the heart, which is executed before setting a scan parameter 28 (refer to FIG. 2) to be described below, or the like, and a start operation of the main imaging gated with the cardiac rest period, which is executed based on the scan parameter 28.

### Function of Control Device

FIG. 2 is a functional block diagram of the control device 16 of the first embodiment. As shown in FIG. 2, a function of the control device 16 is implemented by using various processors. The various processors include a central processing unit (CPU), a graphics processing unit (GPU), an application specific integrated circuit (ASIC), a programmable logic device [for example, simple programmable logic devices (SPLD), complex programmable logic device (CPLD), and field programmable gate arrays (FPGA)], and the like. Various functions of the control device 16 may be implemented by one processor or may be implemented by a plurality of processors of the same type or different types.

In addition to the electrocardiograph 9, the measurement unit 14, the display unit 18, and the operation unit 20 described above, a storage unit 24 is connected to the control device 16.

The storage unit 24 is provided with a signal storage unit 25 and an image storage unit 26 in addition to a control program (not shown) of the MRI apparatus 10. The signal storage unit 25 stores the NMR signal acquired from the measurement unit 14 by the control device 16 in association with the subject's unique identification information [ID] or the like. A plurality of images 22 reconstructed by the control device 16 are stored in the image storage unit 26 in association with the ID of the subject or the like.

In addition, the storage unit 24 stores the scan parameter 28 (also referred to as an imaging parameter and an MR parameter) of the MRI apparatus 10 in association with the ID of the subject or the like. The scan parameter 28 is a setting value (imaging condition) of the MRI imaging by the MRI apparatus 10 and is determined for each site of the subject. The scan parameter 28 of the present embodiment is a setting value corresponding to the MRI imaging of the heart and includes, for example, information indicating the cardiac rest phase (elapsed time from an R-wave of the electrocardiogram waveform EC to a cardiac rest period). The MRI apparatus 10 executes the MRI imaging (main imaging) gated with the cardiac rest period of at least one of the end-diastole or the end-systole of the pulsating heart based on the scan parameter 28.

The control device 16 executes a control program (not shown) stored in the storage unit 24 to function as a measurement control unit 30, a reconstruction unit 31, a time-phase detection unit 32, a display control unit 33, a time-phase correction unit 34, and a parameter setting unit 35.

The measurement control unit 30 operates in a case where the start operation of the MRI imaging (the cine imaging or the main imaging) is input to the operation unit 20. The measurement control unit 30 controls the measurement unit 14 based on the electrocardiogram waveform EC of the subject input from the electrocardiograph 9 to execute MRI imaging of acquiring nuclear magnetic resonance signals (NMR signals) of the heart for one or more heartbeats in synchronization with the electrocardiogram waveform EC. In the MRI imaging of the present embodiment, the measurement control unit 30 acquires the NMR signals for a period of a plurality of heartbeats.

The measurement control unit 30 executes known retrospective cine imaging in a case where the start operation of the cine imaging is input to the operation unit 20. In the retrospective cine imaging, the electrocardiogram waveform EC is stored in the signal storage unit 25 together with the NMR signals, and the reconstruction unit 31 to be described below rearranges the NMR signals in a post-processing to reconstruct the image 22. The measurement control unit 30 may execute known prospective cine imaging of acquiring the NMR signal in synchronization with the R-wave of the electrocardiogram waveform EC, instead of executing the retrospective cine imaging.

In addition, in a case where the start operation of the main imaging is input to the operation unit 20, the measurement control unit 30 controls the measurement unit 14 based on the electrocardiogram waveform EC of the subject and the scan parameter 28 in the storage unit 24 to execute the acquisition of the NMR signal gated with the cardiac rest period of the subject.

The reconstruction unit 31 performs reconstruction of the image 22 by using a known method based on the NMR signal and the like stored in the signal storage unit 25. Specifically, in a case where the retrospective cine imaging is executed by the measurement control unit 30, the reconstruction unit 31 reconstructs the plurality of images 22 along a time series spanning one heartbeat of the heart at regular time intervals (including substantially regular time intervals), based on the NMR signals and the electrocardiogram waveform EC for a plurality of heartbeats. The time interval of the images 22 can be adjusted as appropriate.

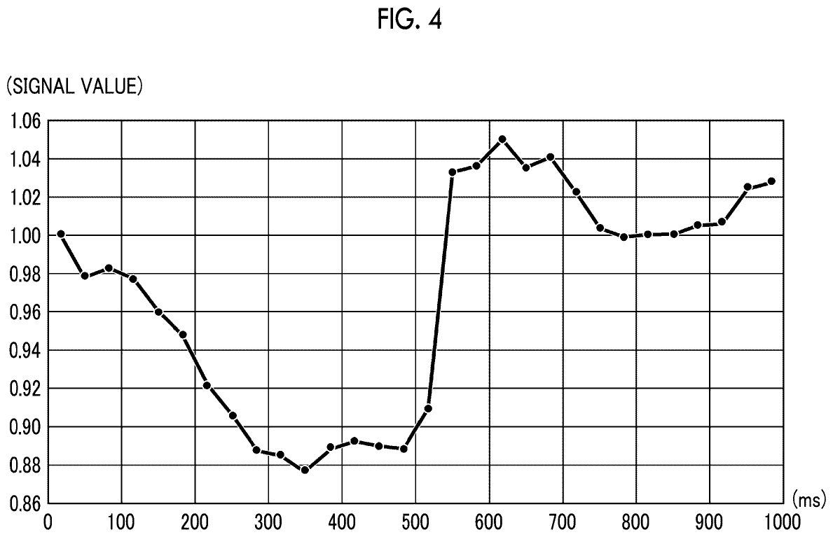

For example, in the present embodiment, the reconstruction unit 31 reconstructs the image 22 at each of regular time intervals in which one heartbeat (approximately 1000 ms) of the heart is divided into 30 intervals, based on the NMR signals acquired for a period of 10 heartbeats (refer to FIG. 4). Each image 22 (hereinafter, referred to as a time series image group) reconstructed by the reconstruction unit 31 is used for the detection of the cardiac rest phase by the time-phase detection unit 32 to be described below because time phases (the elapsed time from the R-wave of the electrocardiogram waveform EC) of individual images 22 are known. In addition, the individual images 22 of the time series image group are also used as diagnostic images for diagnosis. Then, the reconstruction unit 31 stores the time series image group in the image storage unit 26 in association with the ID of the subject or the like.

In addition, in a case where the main imaging is executed by the measurement control unit 30, the reconstruction unit 31 reconstructs the image 22 based on the NMR signal acquired in accordance with the cardiac rest period of the subject and stores the image 22 in the image storage unit 26 in association with the ID of the subject or the like. Further, the reconstruction unit 31 can reconstruct the image 22 by selecting an NMR signal corresponding to the cardiac rest period from among the NMR signals that have been acquired in the retrospective cine imaging.

The time-phase detection unit 32 detects one or more cardiac rest phases based on the time series image group stored in the image storage unit 26. Here, the cardiac rest phase includes an elapsed time (start phase) from the R-wave to the start of the cardiac rest period and an elapsed time (end phase) from the R-wave to the end of the cardiac rest period. As shown in FIG. 4 to be described below, the time-phase detection unit 32 detects a first cardiac rest phase P1 indicating an end-systolic cardiac rest phase of the heart and a second cardiac rest phase P2 indicating an end-diastolic cardiac rest phase of the heart, as the cardiac rest phases. Specifically, the time-phase detection unit 32 executes signal value detection processing of detecting a signal value from each image 22 of the time series image group, and rest phase detection processing of detecting the first cardiac rest phase P1 and the second cardiac rest phase P2 based on a detection result of the signal value detection processing.

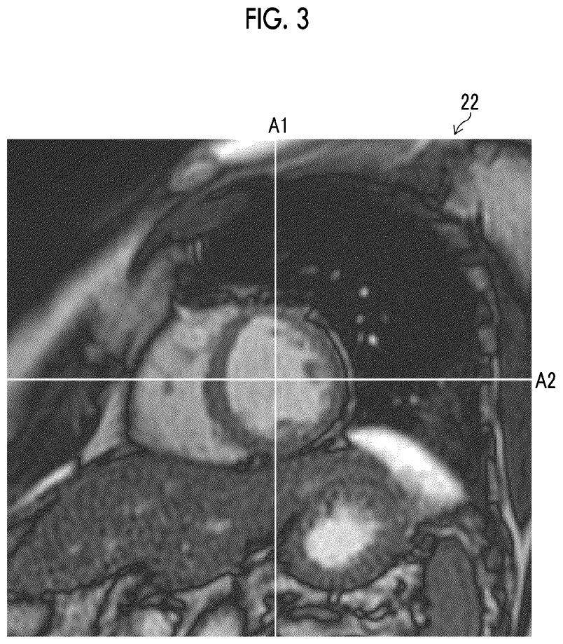

FIG. 3 is an explanatory diagram showing an example of the signal value detection processing by the time-phase detection unit 32. FIG. 4 is a graph showing a relationship between the time phase (horizontal axis) of each image 22 of the time series image group spanning one heartbeat of the heart and the signal value (vertical axis) of each image 22. The signal value of each image 22 in FIG. 4 is standardized with the signal value of the first (immediately after the R-wave) image 22 as a reference.

As shown in FIG. 3, the time-phase detection unit 32 executes the signal value detection processing of detecting the signal value for each image 22 of the time series image group stored in the image storage unit 26. For example, the time-phase detection unit 32 calculates an average value of pixel signal values of pixels on two axes (a vertical axis A1 and a horizontal axis A2) passing through the center (observation visual field center) of the image 22 for each image 22 and detects this average value as the signal value (representative value) of the image 22. The signal value of the image 22 increases as an area occupied by blood increases in the image 22 and conversely, decreases as the area occupied by blood decreases. Therefore, the signal value of the image 22 increases in the diastolic period of the heart and conversely, decreases in the systolic period of the heart. The method of detecting the signal value of each image 22 is not limited to the above-described method, and various known methods are used.

As shown in FIG. 4, the time-phase detection unit 32 executes the signal value detection processing for each image 22, thereby obtaining the relationship between the time phase of the image 22 at each of the regular time intervals spanning one heartbeat of the heart and the signal value for each image 22.

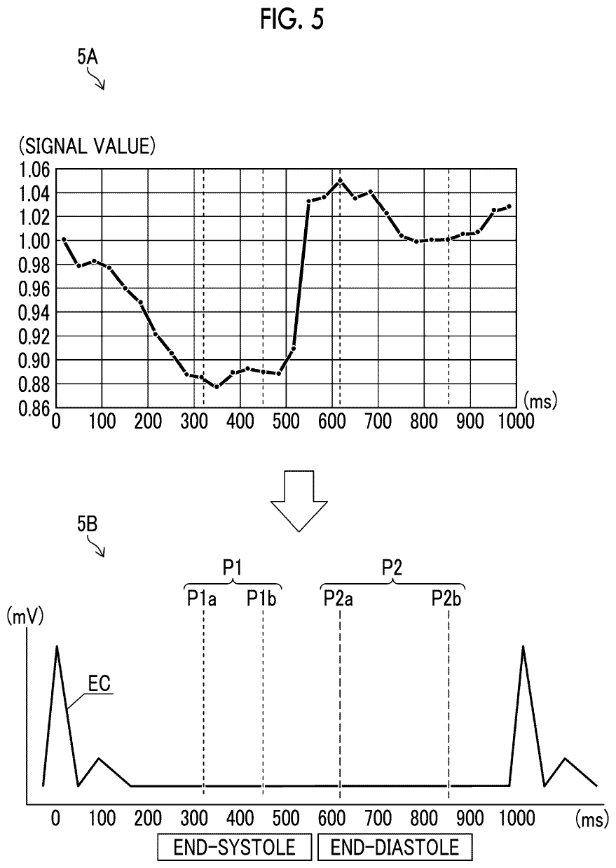

FIG. 5 is an explanatory diagram illustrating an example of the rest phase detection processing by the time-phase detection unit 32. A reference numeral 5A in FIG. 5 indicates a graph shown in FIG. 4, and a reference numeral 5B in FIG. 5 indicates a diagram in which the first cardiac rest phase P1 and the second cardiac rest phase P2 are superimposed and displayed on the electrocardiogram waveform EC.

As shown in FIG. 5, the time-phase detection unit 32 executes the rest phase detection processing of detecting the first cardiac rest phase P1 (a first start phase P1a and a first end phase P1b) and the second cardiac rest phase P2 (a second start phase P2a and a second end phase P2b) based on the relationship between the time phase of the image 22 at each of the regular time intervals spanning one heartbeat of the heart and the signal value for each image 22.

For example, the time-phase detection unit 32 uses a known data analysis method to detect a first range in which the time change of the signal value is small after the signal value has decreased with the elapse of time, and a second range in which the time change of the signal value is small after the signal value has increased with the elapse of time. Then, the time-phase detection unit 32 decides on a part of the first range as the first cardiac rest phase P1 and decides on a part of the second range as the second cardiac rest phase P2. A method of deciding on the first cardiac rest phase P1 and the second cardiac rest phase P2 is not particularly limited. In addition, the detection result of the time-phase detection unit 32 may be stored in the storage unit 24 in association with the ID of the subject or the like.

The time-phase detection unit 32 may detect only one of the first cardiac rest phase P1 or the second cardiac rest phase P2 in accordance with a predetermined setting condition, instead of detecting both the first cardiac rest phase P1 and the second cardiac rest phase P2.

### Time-Phase Setting UI Screen

FIG. 6 is an explanatory diagram showing an example of the time-phase setting UI screen 40 of the first embodiment. As shown in FIG. 6 and FIG. 2 described above, the display control unit 33 generates the time-phase setting UI screen 40 based on the detection results of the first cardiac rest phase P1 and the second cardiac rest phase P2 by the time-phase detection unit 32 and the time series image group stored in the image storage unit 26 and displays the time-phase setting UI screen 40 on the display unit 18. The time-phase setting UI screen 40 includes an image display region 40A and a time-phase display region 40B.

Any of the plurality of images 22 constituting the time series image group is selectively displayed in the image display region 40A. In addition, the image display region 40A includes a time-phase display region 42 and an image selection icon 43.

The time-phase display region 42 displays the time phase (cardiac phase) of the image 22 displayed in the image display region 40A.

The image selection icon 43 is used for the user to perform the image selection operation of selecting the image 22 to be displayed in the image display region 40A from among the plurality of images 22 constituting the time series image group. The image selection operation is, for example, a designation operation (a mouse click operation or the like) of designating the image selection icon 43 with a cursor 41 and is input to the operation unit 20 by the user. The plurality of images 22 may be displayed in the image display region 40A, and a display aspect of the image 22 selected in the image selection operation may be made different from the other images 22.

The time-phase display region 40B includes a detection result field 44. In the detection result field 44, the electrocardiogram waveform EC, and the first cardiac rest phase P1 and the second cardiac rest phase P2 detected by the time-phase detection unit 32 are displayed in accordance with a common time axis T (ms). In addition, a time indicator 45 indicating a time (time phase) on the time axis T corresponding to the image 22 being displayed in the image display region 40A is displayed in the detection result field 44. The time indicator 45 is composed of, for example, a slider bar and a numerical value, but the display aspect thereof is not particularly limited.

The time indicator 45 is used for the user to perform the time designation operation of designating a certain time on the time axis T, as well as to indicate the time (time phase) on the time axis T corresponding to the image 22 being displayed in the image display region 40A. The time designation operation is, for example, a movement operation (a drag operation or the like) of moving the slider bar to a certain time on the time axis T by using the cursor 41 and is input to the operation unit 20 by the user.

In addition, the time-phase display region 40B includes a first start phase display field 46, a first end phase display field 47, a second start phase display field 48, a second end phase display field 49, a first selection field 50, a second selection field 51, a setting icon 52, and the like.

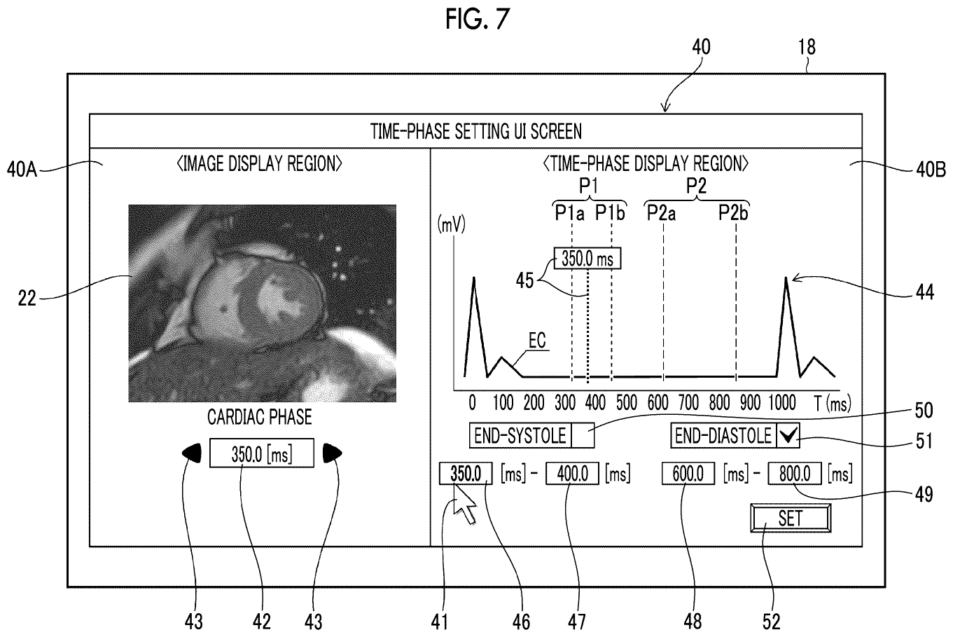

The first start phase P1a is displayed in the first start phase display field 46, and the first end phase P1b is displayed in the first end phase display field 47. The second start phase P2a is displayed in the second start phase display field 48, and the second end phase P2b is displayed in the second end phase display field 49. The time phases respectively displayed in the first start phase display field 46, the first end phase display field 47, the second start phase display field 48, and the second end phase display field 49 can be corrected by the correction operation (refer to FIG. 7 to be described below). This correction operation is, for example, a numerical value input operation for the user to input a new time phase into a time-phase display field to be corrected and is input to the operation unit 20 by the user.

The first selection field 50 is used for the time-phase selection operation of selecting the setting of the scan parameter 28 based on the first cardiac rest phase P1. In addition, the second selection field 51 is used for the time-phase selection operation of selecting the setting of the scan parameter 28 based on the second cardiac rest phase P2. These time-phase selection operations are, for example, designation operations (mouse click operations or the like) for the user to respectively designate the first selection field 50 and the second selection field 51 by using the cursor 41 and are input to the operation unit 20 by the user. FIG. 6 shows a case where the setting of the scan parameter 28 based only on the second cardiac rest phase P2 is executed as an example.

By performing the time-phase selection operation for only one of the first selection field 50 or the second selection field 51, any one of the first cardiac rest phase P1 or the second cardiac rest phase P2 can be selected for the setting of the scan parameter 28.

The setting icon 52 is used for the scan parameter setting operation of executing the setting of the scan parameter 28 by the parameter setting unit 35 to be described below by finalizing the first cardiac rest phase P1 and the second cardiac rest phase P2. The scan parameter setting operation is, for example, a designation operation (a mouse click operation or the like) of designating the setting icon 52 and is input to the operation unit 20 by the user.

### Image Selection Operation

In a case where the image selection operation for the image selection icon 43 is input to the operation unit 20, the display control unit 33 acquires the image 22 selected in the image selection operation from the time series image group stored in the image storage unit 26 and displays the acquired image 22 in the image display region 40A. In addition, the display control unit 33 displays the time phase of the image 22 selected in the image selection operation in the time-phase display region 42.

Further, the display control unit 33 changes the display of the time indicator 45 according to the time phase of the image 22 selected in the image selection operation. Specifically, the display control unit 33 changes the display position of the slider bar of the time indicator 45 on the time axis T and the numerical value of the time indicator 45 in accordance with the time phase of the image 22 selected in the image selection operation. As a result, the time (time phase) of the image 22 selected in the image selection operation is displayed on the time axis T of the detection result field 44.

### Time Designation Operation

In a case where the time designation operation for the time indicator 45 (slider bar) is input to the operation unit 20, the display control unit 33 acquires the image 22 corresponding to the time (time phase) indicated by the time indicator 45 after the time designation operation from the time series image group stored in the image storage unit 26 and displays the acquired image 22 in the image display region 40A. In addition, the display control unit 33 displays the time (time phase) indicated by the time indicator 45 after the time designation operation in the time-phase display region 42. As a result, the image 22 corresponding to a certain time designated in the time designation operation can be displayed in the image display region 40A.

### Correction Operation

By the above-described time designation operation, the images 22 respectively corresponding to the first start phase P1a, the first end phase P1b, the second start phase P2a, and the second end phase P2b detected by the time-phase detection unit 32 can be selectively displayed in the image display region 40A. Consequently, the user can determine whether or not “the first start phase P1a and the first end phase P1b detected by the time-phase detection unit 32” are different from “the actual first start phase P1a and the actual first end phase P1b” based on the images 22 corresponding to the first start phase P1a and the first end phase P1b. In addition, similarly, the user can determine whether or not “the second start phase P2a and the second end phase P2b detected by the time-phase detection unit 32” are different from “the actual second start phase P2a and the actual second end phase P2b” based on the images 22 corresponding to the second start phase P2a and the second end phase P2b.

FIG. 7 is an explanatory diagram showing an example of the time-phase setting UI screen 40 after the correction operation. As shown in FIG. 7, for example, in a case where the first start phase P1a detected by the time-phase detection unit 32 is different from the actual first start phase P1a, the user selects the image 22 suitable for the actual first start phase P1a while switching the images 22 to be displayed in the image display region 40A through the image selection operation described above. Consequently, the time phase corresponding to the image 22, that is, the actual first start phase P1a (here, 350 ms) can be indicated to the user by the time indicator 45. As a result, the user can easily understand the actual first start phase P1a.

For example, in a case where the correction operation for the first start phase display field 46 (first start phase P1a) is input to the operation unit 20, the time-phase correction unit 34 (refer to FIG. 2) corrects the first start phase P1a detected by the time-phase detection unit 32 to the actual first start phase P1a based on the correction operation. In addition, the display control unit 33 corrects the first start phase P1a to be displayed in the first start phase display field 46 in response to the correction by the time-phase correction unit 34.

By using the same method even in a case where the first end phase P1b, the second start phase P2a, and the second end phase P2b detected by the time-phase detection unit 32 are respectively different from the actual time phases, the user understands each of the actual first end phase P1b, the actual second start phase P2a, and the actual second end phase P2b, thereby executing the correction operation.

### Time-Phase Selection Operation

In a case where the time-phase selection operation for any one of the first selection field 50 or the second selection field 51 is input to the operation unit 20, the display control unit 33 changes the display aspect of the one field, for example, displays a check mark. In addition, in a case where the time-phase selection operations for both the first selection field 50 and the second selection field 51 are input to the operation unit 20, the display control unit 33 changes the display aspects of both fields.

### Scan Parameter Setting Operation

The parameter setting unit 35 (refer to FIG. 2) operates in a case where the scan parameter setting operation for the setting icon 52 is input to the operation unit 20, that is, in a case where the first cardiac rest phase P1 and the second cardiac rest phase P2 are finalized. The parameter setting unit 35 performs the setting of the scan parameter 28 based on the setting state of the time-phase setting UI screen 40 and stores the scan parameter 28 in the storage unit 24.

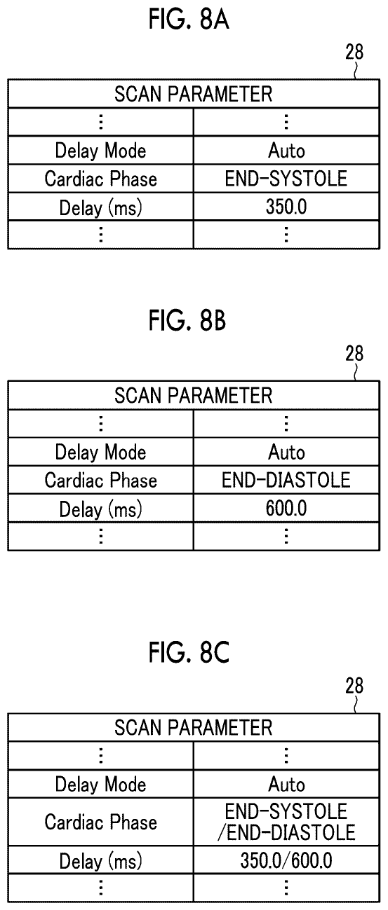

FIGS. 8A to 8C are explanatory diagrams showing examples of the scan parameter 28. In FIGS. 8A to 8C, among various setting items (imaging conditions) of the scan parameter 28, only setting items (“delay mode”, “cardiac phase”, and “delay [ms]”) related to the start condition of the MRI imaging are shown, and other setting items are not shown.

“Auto” is set in the “delay mode” of the scan parameter 28 in a case where the scan parameter 28 is automatically set by the parameter setting unit 35, and “manual” is set in a case where the scan parameter 28 is manually set by the user.

In the “cardiac phase” of the scan parameter 28, “end-systole” is set in a case where the MRI imaging gated with the end-systolic cardiac rest period is performed, “end-diastole” is set in a case where the MRI imaging gated with the end-diastolic cardiac rest period is performed, and “end-systole/end-diastole” is set in a case where the MRI imaging gated with both the end-systolic cardiac rest period and the end-diastolic cardiac rest period is performed.

In the “delay [ms]” of the scan parameter 28, a delay time of the MRI imaging start with respect to the R-wave as a reference, that is, at least one of the first start phase P1a or the second start phase P2a is set.

As shown in FIG. 8A, in a case where only the first selection field 50 is selected in the time-phase selection operation, the parameter setting unit 35 sets the “delay mode” of the scan parameter 28 to “auto” and sets the “cardiac phase” to “end-systole”. In addition, the parameter setting unit 35 sets the first start phase P1a (here, 350.0 ms) in the first start phase display field 46 as “delay [ms]”. As a result, the first start phase P1a is set as “delay [ms]” as it is in a case where the first start phase P1a is not subjected to the correction operation, and the first start phase P1a after the correction operation is set as “delay [ms]” in a case where the first start phase P1a is subjected to the correction operation.

As shown in FIG. 8B, in a case where only the second selection field 51 is selected in the time-phase selection operation, the parameter setting unit 35 sets the “delay mode” of the scan parameter 28 to “auto” and sets the “cardiac phase” to “end-diastole”. In addition, the parameter setting unit 35 sets the second start phase P2a (here, 600.0 ms) in the second start phase display field 48 as “delay [ms]”. As a result, the second start phase P2a is set as “delay [ms]” as it is in a case where the second start phase P2a is not subjected to the correction operation, and the second start phase P2a after the correction operation is set as “delay [ms]” in a case where the second start phase P2a is subjected to the correction operation.

As shown in FIG. 8C, in a case where both the first selection field 50 and the second selection field 51 are selected in the time-phase selection operation, the parameter setting unit 35 sets the “delay mode” of the scan parameter 28 to “auto” and sets the “cardiac phase” to “end-systole/end-diastole”. In addition, the parameter setting unit 35 sets the first start phase P1a in the first start phase display field 46 and the second start phase P2a in the second start phase display field 48 as “delay [ms]”.

Although not shown, the parameter setting unit 35 also sets the MRI imaging end time for the scan parameter 28 based on the first end phase P1b input to the first end phase display field 47 of the time-phase setting UI screen 40 and the second end phase P2b input to the second end phase display field 49.

In addition, the parameter setting unit 35 may select any one of the first cardiac rest phase P1 or the second cardiac rest phase P2 based on a predetermined recommendation condition, regardless of the time-phase selection operation by the user, and may set the scan parameter 28 based on the selected one. This recommendation condition is, for example, determined based on a facility policy or determined based on a heart rate of the subject detected by the electrocardiograph 9. Information regarding this recommendation condition is stored in advance in the storage unit 24 or the like. The setting content of the recommendation condition can be changed by operating the operation unit 20.

### Re-Correction Operation

The scan parameter 28 can be corrected even after being set by the parameter setting unit 35. In this case, the display control unit 33 re-displays the time-phase setting UI screen 40 on the display unit 18 in response to an input of a display operation of the time-phase setting UI screen 40 for the operation unit 20. Consequently, the user inputs the re-correction operation similar to the correction operation performed before the setting of the scan parameter 28 to the operation unit 20, thereby executing the re-correction of the first start phase P1a, the first end phase P1b, the second start phase P2a, and the second end phase P2b by the time-phase correction unit 34. In addition, the display control unit 33 updates the display of the first start phase display field 46, the first end phase display field 47, the second start phase display field 48, and the second end phase display field 49 in response to the re-correction. The re-correction operations of the first start phase P1a, the first end phase P1b, the second start phase P2a, and the second end phase P2b can be executed a plurality of times.

In addition, even in a case where the correction operation is not performed before the setting of the scan parameter 28, the correction operations of the first start phase P1a, the first end phase P1b, the second start phase P2a, and the second end phase P2b initially detected by the time-phase detection unit 32 can be executed at least once. In this case as well, the correction by the time-phase correction unit 34 and the display update by the display control unit 33 are executed.

In a case where the scan parameter setting operation for the time-phase setting UI screen 40 (setting icon 52) re-displayed on the display unit 18 is input to the operation unit 20, the parameter setting unit 35 resets (corrects) the scan parameter 28 stored in the storage unit 24 based on the setting state of the time-phase setting UI screen 40 after the correction by the time-phase correction unit 34.

### Action of First Embodiment

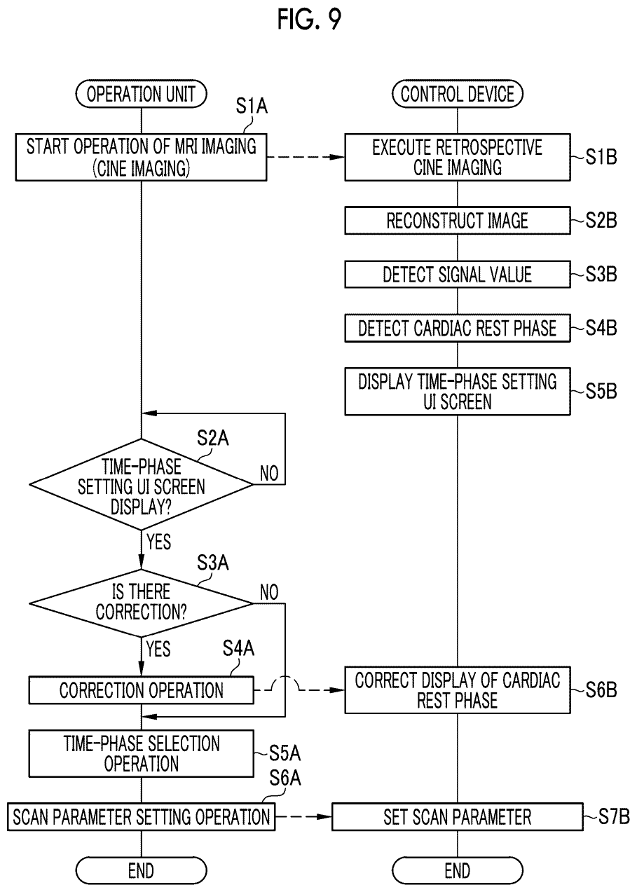

FIG. 9 is a flowchart showing a data processing method of the MRI apparatus 10 of the first embodiment, particularly a flow of scan parameter settings. As shown in FIG. 9, in a case where the user inputs the start operation of the MRI imaging (cine imaging) to the operation unit 20 (step S1A), the control device 16 functions as the measurement control unit 30, the reconstruction unit 31, the time-phase detection unit 32, the display control unit 33, the time-phase correction unit 34, and the parameter setting unit 35.

First, the measurement control unit 30 controls the measurement unit 14 based on the electrocardiogram waveform EC of the subject input from the electrocardiograph 9 to execute the retrospective cine imaging (electrocardiogram gated imaging) of the heart for the period of the plurality of heartbeats (step S1B). Consequently, the NMR signals acquired by the measurement unit 14 and the electrocardiogram waveform EC of the electrocardiograph 9 are stored in the signal storage unit 25. In this way, by acquiring data (the NMR signals and the electrocardiogram waveform EC) for the period of the plurality of heartbeats, heart rate variations in a data acquisition period are averaged unlike the related art in which the data acquisition period is only one heartbeat, and time phase discrepancies caused by the heart rate variations during the examination can be reduced.

In a case where the retrospective cine imaging is completed, the reconstruction unit 31 reconstructs the time series images 22 spanning one heartbeat of the heart at regular time intervals based on the NMR signals and the electrocardiogram waveform EC for the plurality of heartbeats stored in the signal storage unit 25 (step S2B, corresponding to a reconstruction step of the embodiment of the present invention). Then, the plurality of time-phase images 22 reconstructed by the reconstruction unit 31 at regular time intervals are stored in the image storage unit 26 as the time series image group. Each image 22 can be used as a diagnostic image. In this case, there is no need to perform the MRI imaging again, so that extension of the examination time does not occur.

Next, the time-phase detection unit 32 executes the signal value detection processing of detecting the signal value (representative value) for each image 22 based on the time series image group stored in the image storage unit 26, as shown in FIG. 3 described above (step S3B). Then, as shown in FIGS. 4 and 5 described above, the time-phase detection unit 32 executes the rest phase detection processing of detecting the first cardiac rest phase P1 and the second cardiac rest phase P2 based on the relationship between the time phase of the image 22 at each of the regular time intervals spanning one heartbeat and the signal value (step S4B). Steps S3B and S4B correspond to a time-phase detection step of the embodiment of the present invention.

In a case where the rest phase detection processing is completed, the display control unit 33 generates the time-phase setting UI screen 40 shown in FIG. 6 described above based on the detection result of the time-phase detection unit 32 and the time series image group stored in the image storage unit 26 and displays the time-phase setting UI screen 40 on the display unit 18 (step S5B, corresponding to a display control step of the embodiment of the present invention). As a result, the first cardiac rest phase P1 (the first start phase P1a and the first end phase P1b) and the second cardiac rest phase P2 (the second start phase P2a and the second end phase P2b) are displayed on the display unit 18 in an identifiable manner.

In a case where the time-phase setting UI screen 40 is displayed on the display unit 18 (YES in step S2A), the user determines whether or not the first cardiac rest phase P1 and the second cardiac rest phase P2 in the time-phase setting UI screen 40 are different from the actual first cardiac rest phase P1 and the actual second cardiac rest phase P2.

For example, the user inputs the time designation operation described above to the operation unit 20 to display the image 22 corresponding to the first start phase P1a detected by the time-phase detection unit 32 in the image display region 40A. As a result, the user can determine whether or not the first start phase P1a detected by the time-phase detection unit 32 is different from the actual first start phase P1a, that is, whether or not the correction operation of the first start phase P1a is necessary, based on the image 22 displayed in the image display region 40A (step S3A).

In a case where the user determines that the correction operation of the first start phase P1a is necessary (YES in step S3A), for example, the user inputs the image selection operation described above to the operation unit 20 to select the image 22 suitable for the actual first start phase P1a while switching the images 22 to be displayed in the image display region 40A. Consequently, the display control unit 33 adjusts the position and the numerical value of the slider bar of the time indicator 45 based on the time (time phase) corresponding to the image 22 selected by the user. As a result, the user can understand the actual first start phase P1a.

After understanding the actual first start phase P1a, the user inputs the correction operation of the first start phase P1a to the operation unit 20 (step S4A, corresponding to an input acceptance step of the embodiment of the present invention). Consequently, the time-phase correction unit 34 corrects the first start phase P1a detected by the time-phase detection unit 32 to the actual first start phase P1a, and the display control unit 33 corrects the display of the first start phase display field 46 in response to the correction (step S6B). Step S6B corresponds to a time-phase correction step of the embodiment of the present invention.

As described above, in the present embodiment, by juxtaposing and displaying the image display region 40A and the time-phase display region 40B in the time-phase setting UI screen 40, the user can perform the above-described time designation operation and image selection operation while referring to both regions. As a result, the user can intuitively determine the necessity of the correction operation of the first start phase P1a and intuitively understand the actual first start phase P1a, so that the correction of the first start phase P1a can be easily executed.

Similarly, the user also determines the necessity of the correction operation for each of the remaining first end phase P1b, second start phase P2a, and second end phase P2b, and understands the actual time phase and executes the correction operation in a case where the correction operation is necessary.

In a case where all the necessary correction operations are completed or in a case where the correction operation is not necessary (NO in step S3A), the user inputs the time-phase selection operation described above to the operation unit 20 (step SSA). Consequently, at least one of the first cardiac rest phase P1 or the second cardiac rest phase P2 is selected for the setting of the scan parameter 28. The time-phase selection operation (step S5A) may be executed before the correction operation (step S4A). In addition, as described above, in a case where the recommendation condition for the first cardiac rest phase P1 and the second cardiac rest phase P2 is set in advance, the time-phase selection operation by the user can be omitted.

Next, the user inputs the scan parameter setting operation to the operation unit 20 (step S6A). Consequently, the first cardiac rest phase P1 and the second cardiac rest phase P2 are finalized. Then, as shown in FIGS. 8A to 8C described above, the parameter setting unit 35 executes the setting of the scan parameter 28 based on at least one of the first cardiac rest phase P1 or the second cardiac rest phase P2 in accordance with the selection result of the time-phase selection operation or the recommendation condition and stores the scan parameter 28 in the storage unit 24 (step S7B).

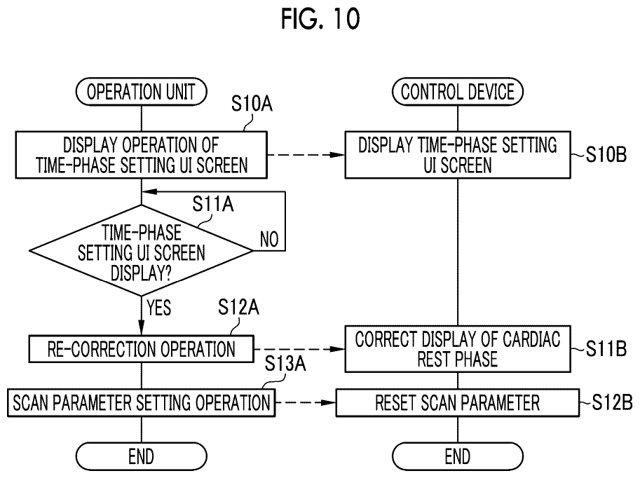

FIG. 10 is a flowchart showing a flow of the re-correction operations of the first cardiac rest phase P1 and the second cardiac rest phase P2 after the setting of the scan parameter 28.

As shown in FIG. 10, in a case where the state of the subject is changed or in a case where the readjustment of the scan parameter 28 is necessary after the MRI imaging (main imaging), the user inputs the display operation of the time-phase setting UI screen 40 to the operation unit 20 (step S10A). In response to this display operation, the display control unit 33 re-displays the time-phase setting UI screen 40 on the display unit 18 (step S10B).

For example, in a case where the first start phase P1a needs to be re-corrected after the time-phase setting UI screen 40 is displayed on the display unit 18 (YES in step S11A), the user inputs the above-described time designation operation or image selection operation to the operation unit 20 while referring to both the image display region 40A and the time-phase display region 40B and decides on an appropriate first start phase P1a. Next, the user inputs the re-correction operation of the first start phase P1a to the operation unit 20 (step S12A). Consequently, the time-phase correction unit 34 re-corrects the first start phase P1a based on the re-correction operation, and the display control unit 33 corrects the display of the first start phase display field 46 (step S11B). As a result, the previously finalized first start phase P1a is re-corrected to an appropriate first start phase P1a.

Similarly, in a case where the re-correction operation is necessary for each of the remaining first end phase P1b, second start phase P2a, and second end phase P2b, the user understands an appropriate time phase and executes the re-correction operation. In addition, the user inputs the time-phase selection operation to the operation unit 20 as necessary.

Then, in a case where the user inputs the scan parameter setting operation to the operation unit 20 (step S13A), the first cardiac rest phase P1 and the second cardiac rest phase P2 are finalized again. Then, the parameter setting unit 35 resets the scan parameter 28 stored in the storage unit 24 (step S12B).

As described above, in the MRI apparatus 10 of the first embodiment, by displaying the time-phase setting UI screen 40 on the display unit 18, the correction operations (re-correction operations) of the first cardiac rest phase P1 and the second cardiac rest phase P2 are configured to be executed on the time-phase setting UI screen 40, so that the user can easily execute the correction (adjustment) of the first cardiac rest phase P1 and the second cardiac rest phase P2. As a result, better accuracy in setting the scan parameter 28 than in the conventional method leads to obtaining high-quality image 22 in the MRI imaging (main imaging).

Further, the reconstruction unit 31 can reconstruct the image 22 for diagnosis by selecting the NMR signal corresponding to the cardiac rest period from among the NMR signals that have been acquired in the retrospective cine imaging, based on the first cardiac rest phase P1 and the second cardiac rest phase P2 after the correction operation. As a result, there is no need to perform the MRI imaging again, so that extension of the examination time does not occur.

### Second Embodiment

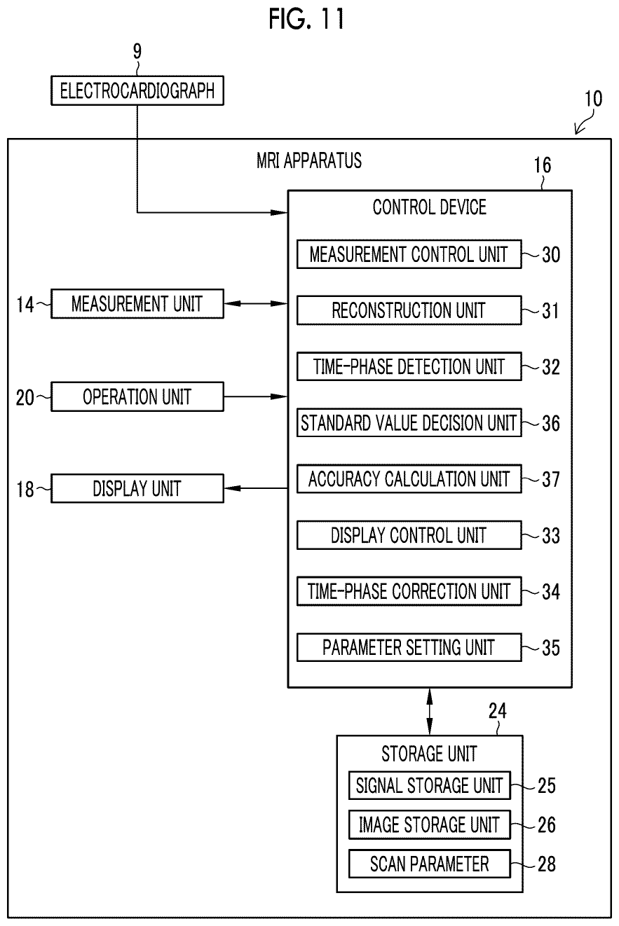

FIG. 11 is a functional block diagram of the control device 16 of the MRI apparatus 10 of the second embodiment. The MRI apparatus 10 of the second embodiment displays accuracy of the first cardiac rest phase P1 (first start phase P1a and first end phase P1b) and the second cardiac rest phase P2 (second start phase P2a and second end phase P2b) on the time-phase setting UI screen 40.

As shown in FIG. 11, the MRI apparatus 10 of the second embodiment has basically the same configuration as the MRI apparatus 10 of the first embodiment, except that the control device 16 functions as a standard value decision unit 36 and an accuracy calculation unit 37. Therefore, the same reference numerals are assigned to the same components as those of the MRI apparatus 10 of the first embodiment in terms of functions or configurations, and descriptions thereof will be omitted.

The standard value decision unit 36 detects the heart rate of the subject based on the electrocardiogram waveform EC of the subject obtained from the electrocardiograph 9 and decides on standard values of the first cardiac rest phase P1 and the second cardiac rest phase P2 corresponding to the heart rate based on a detection result of the heart rate. For example, the standard value decision unit 36 acquires, in advance, correspondence information indicating a relationship between the heart rate and the standard values of the first cardiac rest phase P1 and the second cardiac rest phase P2. As a result, the standard value decision unit 36 decides on the standard values of the first cardiac rest phase P1 and the second cardiac rest phase P2 by referring to the correspondence information based on the detection result of the heart rate of the subject.

The accuracy calculation unit 37 calculates the accuracy of the first cardiac rest phase P1 and the second cardiac rest phase P2 detected by the time-phase detection unit 32 based on the standard values of the first cardiac rest phase P1 and the second cardiac rest phase P2 decided on by the standard value decision unit 36.

For example, the accuracy calculation unit 37 calculates the accuracy to be higher as the difference (discrepancy amount) between the standard value of the first cardiac rest phase P1 decided on by the standard value decision unit 36 and the first cardiac rest phase P1 detected by the time-phase detection unit 32 decreases, and conversely, calculates the accuracy to be lower as the difference increases. In addition, similarly, the accuracy calculation unit 37 calculates the accuracy to be higher as the difference between the standard value of the second cardiac rest phase P2 decided on by the standard value decision unit 36 and the second cardiac rest phase P2 detected by the time-phase detection unit 32 decreases, and conversely, calculates the accuracy to be lower as the difference increases.

The method of calculating the accuracy of the first cardiac rest phase P1 and the second cardiac rest phase P2 is not limited to the above-described method. For example, a trained model generated by using a known machine learning algorithm (a multiple regression model or the like) may be used to calculate the accuracy of the first cardiac rest phase P1 and the second cardiac rest phase P2 detected by the time-phase detection unit 32. The trained model receives “the heart rate of the subject (electrocardiogram waveform EC) and the detection result of the time-phase detection unit 32” as explanatory variables and outputs “the accuracy of the first cardiac rest phase P1 and the second cardiac rest phase P2” as response variables. As a result, the accuracy calculation unit 37 can calculate the accuracy of the first cardiac rest phase P1 and the second cardiac rest phase P2 by inputting the heart rate of the subject and the detection result of the time-phase detection unit 32 to the trained model.

In addition, the accuracy of the first start phase P1a and the accuracy of the first end phase P1b of the first cardiac rest phase P1 may be individually calculated, and the accuracy of the second start phase P2a and the accuracy of the second end phase P2b of the second cardiac rest phase P2 may be individually calculated.

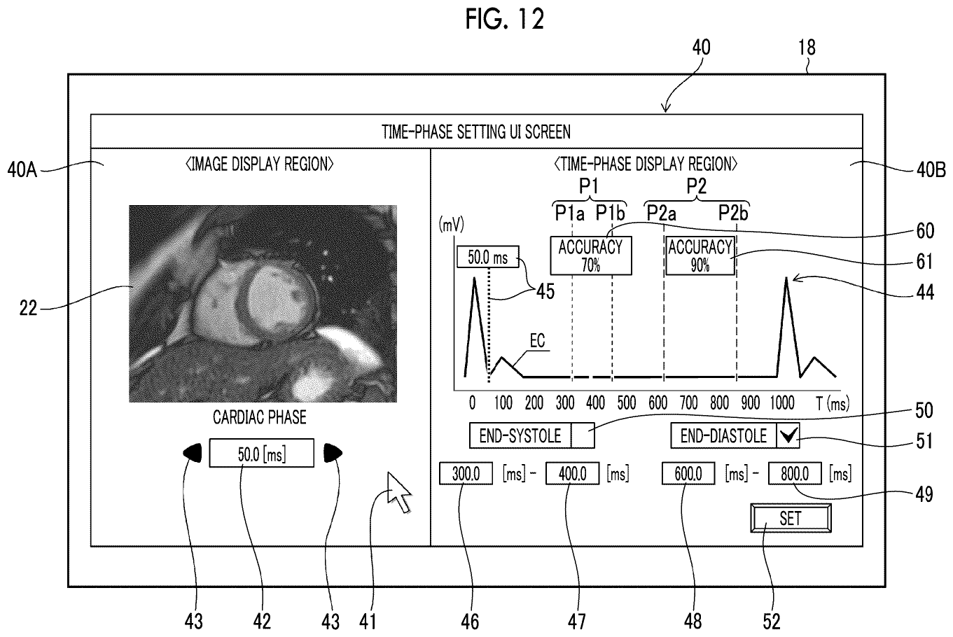

FIG. 12 is an explanatory diagram showing an example of the time-phase setting UI screen 40 of the second embodiment. As shown in FIG. 12, the display control unit 33 of the second embodiment generates the time-phase setting UI screen 40 based on the detection result of the time-phase detection unit 32, the time series image group stored in the image storage unit 26, and the calculation result of the accuracy calculation unit 37 and displays the time-phase setting UI screen 40 on the display unit 18.

The time-phase setting UI screen 40 of the second embodiment is basically the same as the time-phase setting UI screen 40 of the first embodiment described above, except that the time-phase setting UI screen 40 of the second embodiment includes a first accuracy display region 60 and a second accuracy display region 61 generated by the display control unit 33 based on the calculation result of the accuracy calculation unit 37. The first accuracy display region 60 indicates the accuracy of the first cardiac rest phase P1. The second accuracy display region 61 indicates the accuracy of the second cardiac rest phase P2.

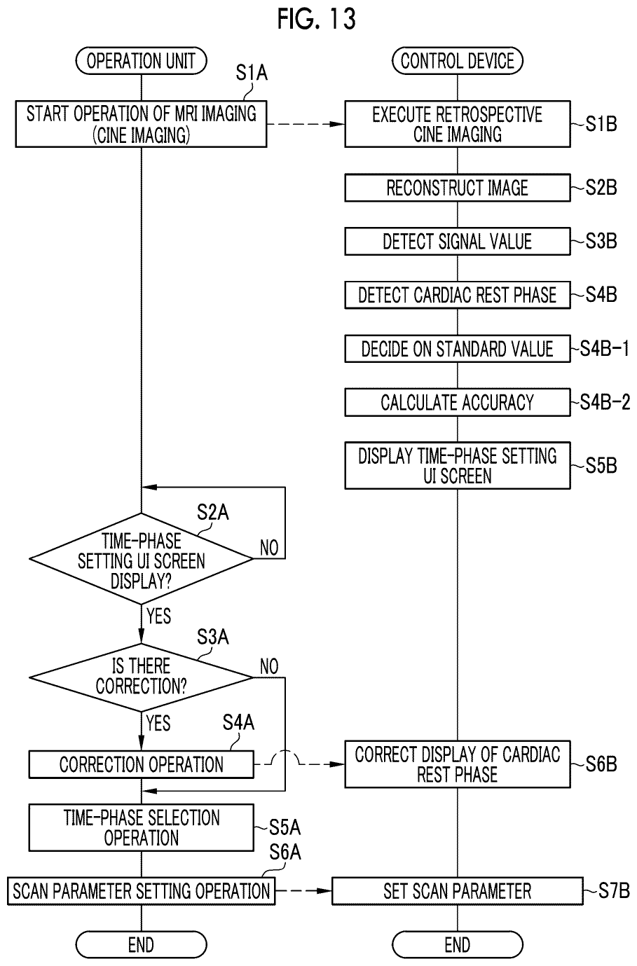

FIG. 13 is a flowchart showing a data processing method of the MRI apparatus 10 of the second embodiment, particularly a flow of scan parameter settings. Since the processing of step S1A in FIG. 13 and the processing from steps S1B to S4B are the same as those of the first embodiment described with reference to FIG. 9 described above, detailed descriptions thereof will be omitted here.

As shown in FIG. 13, in a case where the detection of the first cardiac rest phase P1 and the second cardiac rest phase P2 by the time-phase detection unit 32 is completed (step S4B), the standard value decision unit 36 decides on, based on the heart rate of the subject obtained from the electrocardiogram waveform EC of the electrocardiograph 9, the standard values of the first cardiac rest phase P1 and the second cardiac rest phase P2 corresponding to the heart rate (step S4B-1). Next, the accuracy calculation unit 37 calculates the accuracy of the first cardiac rest phase P1 and the second cardiac rest phase P2 detected by the time-phase detection unit 32 based on the standard values of the first cardiac rest phase P1 and the second cardiac rest phase P2 (step S4B-2).

Then, the display control unit 33 generates the time-phase setting UI screen 40 shown in FIG. 12 described above based on the detection result of the time-phase detection unit 32, the time series image group stored in the image storage unit 26, and the calculation result of the accuracy calculation unit 37 and displays the time-phase setting UI screen 40 on the display unit 18 (step S5B). Since the processing after step S5B is the same as the processing of the first embodiment described with reference to FIG. 9 described above, detailed descriptions thereof will be omitted here.

As described above, in the MRI apparatus 10 of the second embodiment, the accuracy of the first cardiac rest phase P1 and the second cardiac rest phase P2 are calculated and displayed on the display unit 18, so that the user can easily understand the accuracy of the first cardiac rest phase P1 and the second cardiac rest phase P2 even in a case where the user has less experience. As a result, in a case where the accuracy of at least one of the first cardiac rest phase P1 or the second cardiac rest phase P2 is low (for example, in a case where the accuracy is less than a predetermined threshold value), the user can be prompted to re-detect the first cardiac rest phase P1 and the second cardiac rest phase P2 (step S1A, steps S1B to S4B).

### Others

In each of the above-described embodiments, the image display region 40A and the time-phase display region 40B are included in the time-phase setting UI screen 40, but the time-phase setting UI screen 40 may be composed of only the time-phase display region 40B. In this case, it is preferable to display the image 22 on the display unit 18 separately from the time-phase setting UI screen 40.

### EXPLANATION OF REFERENCES

9: electrocardiograph
10: MRI apparatus
14: measurement unit
16: control device
18: display unit
20: operation unit
22: image
24: storage unit
28: scan parameter
30: measurement control unit
31: reconstruction unit
32: time-phase detection unit
33: display control unit
34: time-phase correction unit
35: parameter setting unit
36: standard value decision unit
37: accuracy calculation unit
40: time-phase setting UI screen

What is claimed is:

1. A data processing device of a magnetic resonance imaging apparatus that acquires nuclear magnetic resonance signals of a heart in synchronization with an electrocardiogram waveform for a period of one or more heartbeats, the data processing device comprising:
a reconstruction unit that reconstructs a plurality of images of the heart along a time series based on the nuclear magnetic resonance signals acquired by the magnetic resonance imaging apparatus;
a time-phase detection unit that detects one or more cardiac rest phases based on the plurality of images reconstructed by the reconstruction unit;
a display control unit that displays the cardiac rest phase on a display unit;
an operation unit that accepts an input of a correction operation of the cardiac rest phase; and
a time-phase correction unit that corrects the cardiac rest phase based on the correction operation input to the operation unit.

2. The data processing device according to claim 1, further comprising:
a parameter setting unit that sets a scan parameter of the magnetic resonance imaging apparatus based on the cardiac rest phase,
wherein the parameter setting unit sets, in a case where the correction operation is input to the operation unit, the scan parameter based on the cardiac rest phase corrected by the time-phase correction unit.

3. The data processing device according to claim 2,
wherein the operation unit is configured to accept an input of a re-correction operation of the cardiac rest phase that has been corrected in the correction operation,
the time-phase correction unit re-corrects, in a case where the re-correction operation is input to the operation unit, the cardiac rest phase based on the re-correction operation, and
the parameter setting unit sets, in a case where the re-correction operation is input to the operation unit, the scan parameter based on the cardiac rest phase re-corrected by the time-phase correction unit.

4. The data processing device according to claim 1,
wherein the time-phase detection unit detects a first cardiac rest phase indicating an end-systolic cardiac rest phase of the heart and a second cardiac rest phase indicating an end-diastolic cardiac rest phase of the heart,
the display control unit displays the first cardiac rest phase and the second cardiac rest phase on the display unit in an identifiable manner,
the operation unit accepts inputs of the correction operation of the first cardiac rest phase and the correction operation of the second cardiac rest phase, and
the time-phase correction unit corrects the first cardiac rest phase in a case where the correction operation of the first cardiac rest phase is input to the operation unit, and corrects the second cardiac rest phase in a case where the correction operation of the second cardiac rest phase is input to the operation unit.

5. The data processing device according to claim 4, further comprising:
a parameter setting unit that sets a scan parameter of the magnetic resonance imaging apparatus based on the cardiac rest phase,
wherein the operation unit is configured to accept an input of a time-phase selection operation of selecting any one of the first cardiac rest phase or the second cardiac rest phase, and
the parameter setting unit sets, in a case where the time-phase selection operation is input to the operation unit, the scan parameter based on the one selected in the time-phase selection operation.

6. The data processing device according to claim 4, further comprising:
a parameter setting unit that sets a scan parameter of the magnetic resonance imaging apparatus based on the cardiac rest phase,
wherein the parameter setting unit selects any one of the first cardiac rest phase or the second cardiac rest phase in accordance with a predetermined recommendation condition and sets the scan parameter based on the selected one.

7. The data processing device according to claim 1,
wherein the display control unit displays the cardiac rest phase on the display unit in accordance with a time axis.

8. The data processing device according to claim 7,
wherein the display control unit displays, on the display unit, a time-phase display region including the cardiac rest phase and the time axis, and an image display region for selectively displaying any of the plurality of images reconstructed by the reconstruction unit,
the operation unit is configured to accept an input of a time designation operation of designating a certain time of the time axis, and
the display control unit displays, in a case where the time designation operation is input to the operation unit, an image corresponding to the time designated in the time designation operation among the plurality of images reconstructed by the reconstruction unit, in the image display region.

9. The data processing device according to claim 7,
wherein the display control unit displays, on the display unit, a time-phase display region including the cardiac rest phase and the time axis, and an image display region for selectively displaying any of the plurality of images reconstructed by the reconstruction unit,
the operation unit is configured to accept an input of an image selection operation of selecting the image to be displayed in the image display region, and
the display control unit displays, in a case where the image selection operation is input to the operation unit, the image selected in the image selection operation in the image display region and displays a time phase corresponding to the image selected in the image selection operation on the time axis in the time-phase display region.

10. The data processing device according to claim 1, further comprising:
a standard value decision unit that decides on, based on a heart rate obtained from the electrocardiogram waveform, a standard value of the cardiac rest phase corresponding to the heart rate; and
an accuracy calculation unit that calculates accuracy of the cardiac rest phase detected by the time-phase detection unit based on the standard value decided on by the standard value decision unit,
wherein the display control unit displays a calculation result of the accuracy calculation unit on the display unit.

11. The data processing device according to claim 1,
wherein the reconstruction unit reconstructs the plurality of images at regular time intervals.

12. The data processing device according to claim 1,
wherein the time-phase detection unit calculates a representative value of signal values of the image for each of the plurality of images reconstructed by the reconstruction unit to detect the cardiac rest phase based on the representative value for each image along the time series.

13. A magnetic resonance imaging apparatus comprising:
a measurement control unit that acquires nuclear magnetic resonance signals of a heart in synchronization with an electrocardiogram waveform for a period of one or more heartbeats; and
the data processing device according to claim 1.

14. A data processing method of a magnetic resonance imaging apparatus that acquires nuclear magnetic resonance signals of a heart in synchronization with an electrocardiogram waveform for a period of one or more heartbeats, the data processing method comprising:
a reconstruction step of reconstructing a plurality of images of the heart along a time series based on the nuclear magnetic resonance signals acquired by the magnetic resonance imaging apparatus;
a time-phase detection step of detecting one or more cardiac rest phases based on the plurality of images reconstructed in the reconstruction step;
a display control step of displaying the cardiac rest phase on a display unit;
an input acceptance step of accepting an input of a correction operation of the cardiac rest phase; and
a time-phase correction step of correcting the cardiac rest phase based on the correction operation accepted in the input acceptance step.
